(19) United States
(12) Patent Application Publication
(10) Pub. No.: US 2011/0158914 A1
(43) Pub. Date: Jun. 30, 2011

(54) FLUORESCENCE IMAGE CAPTURING METHOD AND APPARATUS

(75) Inventor: Hideyuki YAMADA, Saitama-shi (JP)

(73) Assignee: FUJIFILM Corporation, Tokyo (JP)

(21) Appl. No.: 12/968,083

(22) Filed: Dec. 14, 2010

(30) Foreign Application Priority Data
Dec. 25, 2009 (JP) 295240/2009

(51) Int. Cl. A61K 49/00; A61B 6/00

(52) U.S. Cl. 424/9.6; 600/476

(57) ABSTRACT

A fluorescence image capturing apparatus for capturing a fluorescence image by receiving fluorescence emitted from an observation area administered with a fluorescent agent, in which pharmacokinetic information of the fluorescent agent is obtained and the intensity of the excitation light and/or the charge storage period of the image sensor is controlled based on the pharmacokinetic information of the fluorescent agent.

### BACKGROUND OF THE INVENTION

1. Field of the Invention

The present invention relates to a fluorescence image capturing method and apparatus for capturing a fluorescence image by directing excitation light to an observation area administered with a fluorescent agent and receiving fluorescence emitted from the fluorescent agent in the observation area.

2. Description of the Related Art

Endoscope systems for observing tissues of body cavities are widely known and an electronic endoscope system that captures an ordinary image of an observation area in a body cavity by directing white light to the observation area and displaying the captured ordinary image on a monitor screen is widely used.

As one type of such endoscope systems described above, a fluorescence endoscope system that obtains an autofluorescence image by directing excitation light to an observation area and capturing an image of autofluorescence emitted from the observation area, in addition to an ordinary image, and displays these images on a monitor screen is proposed as described, for example, in Japanese Unexamined Patent Publication No. 2005-204905.

Further, as one of such fluorescence endoscope systems, a system that obtains a fluorescence image of a blood vessel by administering, for example, indocyanine green into a body in advance and detecting ICG fluorescence in the blood vessel by directing excitation light to the observation area is proposed.

Here, it is known that, when a blood vessel image observation is performed using, for example, the ICG described above, the concentration of the agent in the blood changes by one digit or more by the pharmacokinetics of the agent flowing in the bloodstream. The change in the concentration of the agent causes the intensity of fluorescence of the observation area to be changed largely. This causes a problem of insufficient dynamic range of the image sensor for detecting the fluorescence and a signal detected by the image sensor may be saturated. Further, the time when the intensity of fluorescence in the observation region is changed largely is the time when the fluorescence image is most desired to be captured. Therefore, it is desired that an appropriate fluorescence image is obtained at such timing.

Further, fluorescence emitted from the observation region is very weak and it is necessary to set the charge storage period of an image sensor that photoelectrically converts the weak fluorescence longer than a case in which an ordinary image is captured. But the longer charge storage period causes a problem of blurred fluorescence image due to pulsation of the observation region.

The insufficient dynamic range or the blurring described above causes a problem that the shape or width of a blood vessel can not be recognized accurately.

The present invention has been developed in view of the circumstances described above, and it is an object of the present invention to provide a fluorescence image capturing method and apparatus for capturing a fluorescence image by receiving fluorescence emitted from an observation area administered with a fluorescent agent capable of preventing blurring due to insufficient dynamic range of the image sensor or pulsation of the observation area.

### SUMMARY OF THE INVENTION

A fluorescence image capturing method of the present invention is a method for capturing a fluorescence image by directing excitation light to an observation area of a subject administered with a fluorescent agent, receiving and photoelectrically converting, by an image sensor, fluorescence emitted from the fluorescent agent of the observation area irradiated with the excitation light, and storing charges by the image sensor for a predetermined charge storage period, the method comprising the steps of:

obtaining pharmacokinetic information of the fluorescent agent; and

controlling the intensity of the excitation light and the charge storage period of the image sensor based on the obtained pharmacokinetic information of the fluorescent agent.

A fluorescence image capturing apparatus of the present invention is an apparatus, including:

an excitation light emission unit for emitting excitation light which is directed to an observation area of a subject administered with a fluorescent agent;

an imaging unit having an image sensor for receiving and photoelectrically converting fluorescence emitted from the fluorescent agent of the observation area irradiated with the excitation light and capturing a fluorescence image by storing charges for a predetermined charge storage period by the image sensor;

a pharmacokinetic information obtaining unit for obtaining pharmacokinetic information of the fluorescent agent; and

a control unit for controlling the intensity of the excitation light and the charge storage period of the image sensor based on the pharmacokinetic information of the fluorescent agent obtained by the pharmacokinetic information obtaining unit.

In the fluorescence image capturing apparatus of the present invention described above, the pharmacokinetic information obtaining unit may be a unit that obtains information of a heavily pulsating region of the observation area as the pharmacokinetic information of the fluorescent agent, and the control unit may be a unit that fixes the charge storage period of the image sensor at a relatively short period when the information of a heavily pulsating region is obtained by the pharmacokinetic information obtaining unit.

A fluorescence image capturing apparatus of the present invention is an apparatus, including:

an excitation light emission unit for emitting excitation light which is directed to an observation area of a subject administered with a fluorescent agent;

an imaging unit having an image sensor for receiving and photoelectrically converting fluorescence emitted from the fluorescent agent of the observation area irradiated with the excitation light and capturing a fluorescence image by storing charges for a predetermined charge storage period by the image sensor;

a pharmacokinetic information obtaining unit for obtaining pharmacokinetic information of the fluorescent agent; and

a control unit for controlling the intensity of the excitation light based on the pharmacokinetic information of the fluorescent agent obtained by the pharmacokinetic information obtaining unit.

In the fluorescence image capturing apparatus of the present invention described above, the control unit may a unit that causes the intensity of the excitation light to become relatively small only for a predetermined period based on the pharmacokinetic information of the fluorescent agent.

Further, the pharmacokinetic information obtaining unit may be a unit that obtains an intensity of the excitation light in the predetermined period based on the pharmacokinetic information of the fluorescent agent, and the control unit maybe a unit that controls the intensity of the excitation light based on the intensity of the excitation light in the predetermined period obtained by the pharmacokinetic information obtaining unit.

Still further, the pharmacokinetic information obtaining unit may be a unit that obtains timing for changing the intensity of the excitation light based on the pharmacokinetic information of the fluorescent agent, and the control unit may be a unit that controls the intensity of the excitation light based on the timing obtained by the pharmacokinetic information obtaining unit.

The fluorescence image capturing apparatus described above may further include an assumed fluorescence image signal obtaining unit for obtaining an assumed fluorescence image signal assumed to have been outputted from the image sensor if the excitation light intensity control based on the pharmacokinetic information of the fluorescent agent were not performed based on a fluorescence image signal representing a fluorescence image captured by the imaging unit under the excitation light intensity control by the control unit and information of the excitation light intensity control by the control unit, and an abnormality determination unit for determining an abnormality in the subject based on a change in the assumed fluorescence image signal.

A fluorescence image capturing apparatus of the present invention is an apparatus, including:

an excitation light emission unit for emitting excitation light which is directed to an observation area of a subject administered with a fluorescent agent;

an imaging unit having an image sensor for receiving and photoelectrically converting fluorescence emitted from the fluorescent agent of the observation area irradiated with the excitation light and capturing a fluorescence image by storing charges for a predetermined charge storage period by the image sensor;

a pharmacokinetic information obtaining unit for obtaining pharmacokinetic information of the fluorescent agent; and

a control unit for controlling the charge storage period of the image sensor based on the pharmacokinetic information of the fluorescent agent obtained by the pharmacokinetic information obtaining unit.

In the fluorescence image capturing apparatus of the present invention described above, the control unit may be a unit that causes the charge storage period to become relatively short only for a predetermined period based on the pharmacokinetic information of the fluorescent agent.

Further, the pharmacokinetic information obtaining unit may be a unit that obtains a charge storage period in the predetermined period based on the pharmacokinetic information of the fluorescent agent, and the control unit may be a unit that controls the charge storage period of the image sensor based on the charge storage period obtained by the pharmacokinetic information obtaining unit.

Still further, the pharmacokinetic information obtaining unit may be a unit that obtains timing for changing the charge storage period based on the pharmacokinetic information of the fluorescent agent, and the control unit may be a unit that controls the charge storage period of the image sensor based on the timing obtained by the pharmacokinetic information obtaining unit.

The fluorescence image capturing apparatus of the present invention described above may further includes an assumed fluorescence image signal obtaining unit for obtaining an assumed fluorescence image signal assumed to have been outputted from the image sensor if the charge storage period control based on the pharmacokinetic information of the fluorescent agent were not performed based on a fluorescence image signal representing a fluorescence image captured by the imaging unit under the charge storage period control by the control unit and information of the charge storage period control of the image sensor by the control unit, and an abnormality determination unit for determining an abnormality in the subject based on a change in the assumed fluorescence image signal.

The fluorescence image capturing apparatus of the present invention described above may further include an assumed fluorescence image signal obtaining unit for obtaining an assumed fluorescence image signal assumed to have been outputted from the image sensor if the charge storage period control and the excitation light intensity control based on the pharmacokinetic information of the fluorescent agent were not performed based on a fluorescence image signal representing a fluorescence image captured by the imaging unit under the charge storage period control and the excitation light intensity control by the control unit and information of the charge storage period control of the image sensor and the excitation light intensity control by the control unit, and an abnormality determination unit for determining an abnormality in the subject based on a change in the assumed fluorescence image signal.

Further, the control unit may be a unit that stops or blinks the emission of the excitation light when a predetermined number or more of pixels having a fluorescence image signal greater than or equal to a predetermined threshold value is detected based on a fluorescence image signal of each pixel forming the fluorescence image outputted from the image sensor.

Still further, the pharmacokinetic information of the fluorescent agent may be information that includes information of the subject.

According to the fluorescence image capturing method and apparatus of the present invention, pharmacokinetic information of the fluorescent agent is obtained and the intensity of the excitation light and/or the charge storage period of the image sensor is controlled based on the pharmacokinetic information of the fluorescent agent. This allows, even when the intensity of the fluorescence changes dramatically due to pharmacokinetics of the fluorescent agent, the sensitivity of the image sensor to be changed according to the fluorescence intensity change, whereby the problem of insufficient dynamic range of the image sensor may be prevented. Further, the control of the charge storage period of the image sensor allows a blurred fluorescence image due to pulsation to be prevented.

Further, in the fluorescence image capturing method and apparatus of the present invention, if the timing for changing the intensity of the excitation light is obtained based on the pharmacokinetic information of the fluorescent agent, the charge storage period is allowed to be changed at a time other than a period in which the intensity of the fluorescence changes dramatically due to pharmacokinetics of the fluorescent agent, whereby flame dropping arising from the change in the charge storage period may be prevented from occurring in an important period.

Further, the frame rate may be reduced by setting the charge storage period relatively long during the time other than a period in which the intensity of the fluorescence changes dramatically due to pharmacokinetics of the fluorescent agent, whereby the amount of data to be stored may be reduced and a small capacity memory may be used.

Where information of a heavily pulsating region of the observation area is obtained and the charge storage period is fixed to a relatively short period, a blurred fluorescence image due to pulsation may be prevented.

Further, where an assumed fluorescence image signal assumed to have been outputted from the image sensor if the excitation light intensity control based on the pharmacokinetic information of the fluorescent agent were not performed based on a fluorescence image signal representing a fluorescence image captured by the imaging unit under the excitation light intensity control by the control unit and information of the excitation light intensity control and/or the charge storage period control by the control unit, and an abnormality in the subject is determined based on a change in the in the assumed fluorescence image signal, abnormal information in the subject is allowed to be obtained, as well as the fluorescence image, whereby more valuable information for diagnosis may be obtained.

Still further, where the emission of the excitation light is stopped or blinked when a predetermined number or more of pixels having a fluorescence image signal greater than or equal to a predetermined threshold value is detected based on a fluorescence image signal of each pixel forming the fluorescence image outputted from the image sensor, the observation area may be prevented from being excessively exposed to the excitation light and damaged.

Further, where the pharmacokinetic information of the fluorescent agent includes information of the subject, more appropriate pharmacokinetic information of the fluorescent agent for each subject may be obtained.

### BRIEF DESCRIPTION OF THE DRAWINGS

FIG. 1 is an overview of a rigid endoscope system that employs an embodiment of the fluorescence image capturing apparatus of the present invention.

FIG. 2 is a schematic configuration diagram of the rigid insertion section shown in FIG. 1.

FIG. 3 is a schematic configuration diagram of the imaging unit shown in FIG. 1.

FIG. 4 is a block diagram of the image processing unit and light source unit shown in FIG. 1, illustrating schematic configurations thereof.

FIG. 5 is a schematic configuration diagram of an automatic medicine injector.

FIG. 6 illustrates, by way of example, the intensity variation of a fluorescence image signal obtained in the past.

FIG. 7 illustrates, by way of example, a frame rate and excitation light intensity control information in each period.

FIG. 8 illustrates, by way of example, first derivative values of a fluorescence image signal variation obtained in the past.

FIG. 9 illustrates, by way of example, an ordinary image and a fluorescence image.

FIG. 10 illustrates histograms generated using a fluorescence image signal of each pixel of one frame outputted from a high sensitivity image sensor.

FIG. 11 illustrates, by way of example, an assumed fluorescence image signal.

### DESCRIPTION OF THE PREFERRED EMBODIMENTS

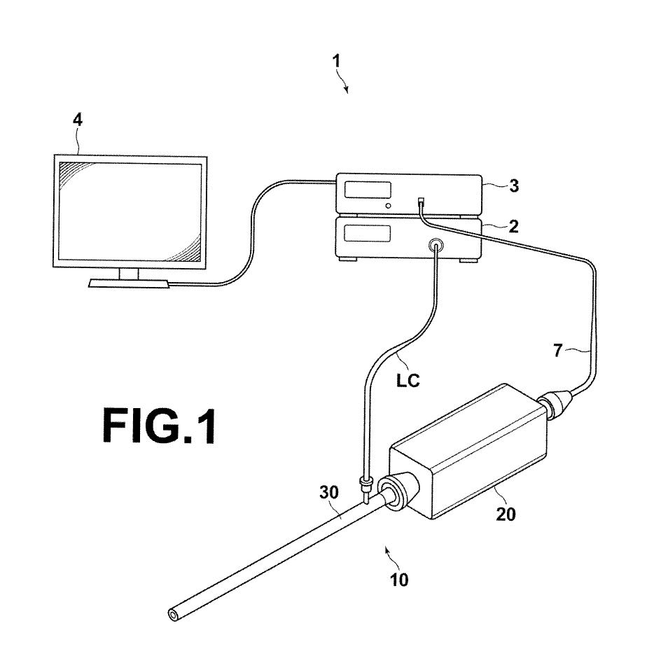

Hereinafter, a rigid endoscope system that employs an embodiment of the fluorescence image capturing apparatus of the present invention will be described in detail with reference to the accompanying drawings. FIG. 1 is an overview of rigid endoscope system 1 of the present embodiment, illustrating a schematic configuration thereof.

As shown in FIG. 1, rigid endoscope system 1 includes light source unit 2 for emitting ordinary light of white light and excitation light, rigid endoscope imaging device 10 for guiding and directing the ordinary light and excitation light emitted from light source unit 2 to an observation area and capturing an ordinary image based on reflection light reflected from the observation area irradiated with the ordinary light and a fluorescence image based on fluorescence emitted from the observation area irradiated with the excitation light, image processing unit 3 for performing predetermined processing on an image signal captured by rigid endoscope imaging device 10, and monitor 4 for displaying an ordinary image and a fluorescence image of the observation area based on a display control signal generated in image processing unit 3.

As shown in FIG. 1, rigid endoscope imaging device 10 includes rigid insertion section 30 to be inserted into a body cavity and imaging unit 20 for capturing an ordinary image and a florescence image of an observation area guided by the rigid insertion section 30.

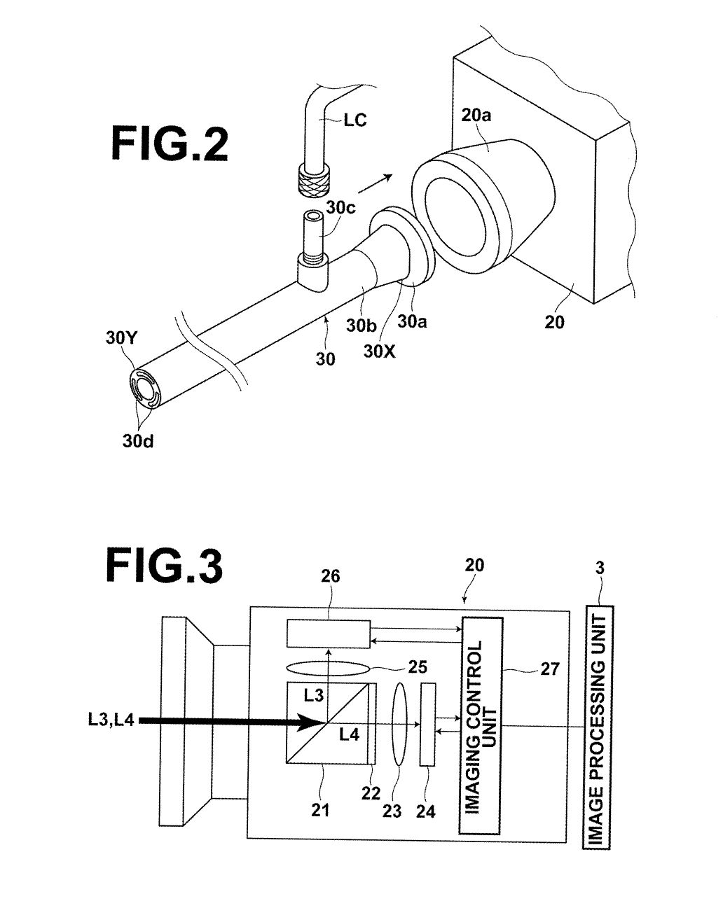

Rigid insertion section 30 and imaging unit 20 are detachably connected, as shown in FIG. 2. Rigid insertion section 30 includes connection member 30a, insertion member 30b, cable connection port 30c, and emission window 30d. Although rigid insertion section 30 and imaging unit 20 are structured so as to be detachably connected in the present embodiment, they may also be integrally formed.

Connection member 30a is provided at first end 30X of rigid insertion section 30 (insertion member 30b), and imaging unit 20 and rigid insertion section 30 are detachably connected by fitting connection member 30a into, for example, aperture 20a formed in imaging unit 20.

Insertion member 30b is a member to be inserted into a body cavity when imaging is performed in the body cavity. Insertion member 30b is formed of a rigid material and has, for example, a cylindrical shape with a diameter of about 10 mm. Insertion member 30b accommodates inside thereof a group of objective lenses for forming an image of an observation area, and an ordinary image and a fluorescence image of the observation area inputted from second end 30Y are inputted, through the group of objective lenses, to imaging unit 20 on the side of first end 30X.

Cable connection port 30c is provided on the side surface of insertion member 30b and an optical cable LC is mechanically connected to the port. This causes light source unit 2 and insertion member 30b to be optically coupled through the optical cable LC.

Emission window 30d is provided on the side of second end 30Y of rigid insertion section 30 to emit ordinary light and excitation light guided through the optical cable LC onto an observation area. Note that a light guide (not shown) for guiding the ordinary light and excitation light from the cable connection port 30c to emission window 30d is provided inside of insertion member 30b, and emission window 30d emits the ordinary light and excitation light guided through the light guide onto the observation area.

FIG. 3 is a schematic configuration diagram of imaging unit 20. Imaging unit 20 includes a first imaging system for generating a fluorescence image signal of an observation area by capturing a fluorescence image of the observation area formed by the group of lenses in rigid insertion section 30 and a second imaging system for generating an ordinary image signal of the observation area by capturing an ordinary image of the observation area formed by the group of lenses in rigid insertion section 30. These imaging systems are divided into two orthogonal optical axes by a dichroic prism 21 having spectroscopic properties in which an ordinary image is reflected and a fluorescence image is transmitted.

The first imaging system includes exitation light cut filter 22 for cutting excitation light reflected from an observation area and transmitted through dichroic prism 21, first image forming system 23 for forming a fluorescence image L4 outputted from rigid insertion section 30 and transmitted through dichroic prism 21 and excitation light cut filter 22, and high sensitivity image sensor 24 for capturing the fluorescence image L4 formed by first image forming system 23.

Second imaging system includes second image forming system 25 for forming an ordinary image L3 outputted from rigid insertion section 30 and reflected by dichroic prism 21, and image sensor 26 for capturing the ordinary image L3 formed by second image forming system 25.

High sensitivity image sensor 24 is a device that detects light in the wavelength range of a fluorescence image with high sensitivity, then converts the detected light to a fluorescence image signal, and outputs the fluorescence image signal. In the present embodiment, a monochrome CCD (charge coupled device) is used as high sensitivity image sensor 24.

Image sensor 26 is a device that detects light in the wavelength range of an ordinary image, then converts the detected light to an ordinary image signal, and outputs the ordinary image signal. In the present embodiment, image sensor 26 is also a CCD image sensor having color filters of three primary colors, red (R), green (G), and blue (B) or of cyan (C), magenta (M), and yellow (Y) arranged in a Beyer or honeycomb pattern on the imaging surface thereof.

Imaging unit 20 further includes imaging control unit 27. Imaging control unit 27 is a unit that performs CDS/AGC (correlated double sampling/automatic gain control) and A/D conversion on a fluorescence image signal outputted from high sensitivity image sensor 24 and an ordinary image signal outputted from image sensor 26, and outputs the resultant image signals to image processing unit 3 through cable 7 (FIG. 1), as well as controlling the operations of high sensitivity image sensor 24 and image sensor 26. Further, imaging control unit 27 is a unit that starts an imaging operation of high sensitivity image sensor based on a signal of depression of foot pedal 6, to be described later.

In the present embodiment, image sensor 26 performs ordinary image capturing at 30 fps, while high sensitivity image sensor 24 is selectable in the frame rate between 10 fps and 20 fps for fluorescence image capturing. Here, the charge storage period of high sensitivity image sensor 24 is also changed according to the selected frame rate. The charge storage period of high sensitivity image sensor 24 at 20 fps is ½ of the charge storage period thereof at 10 fps. The switching timing of the frame rate (charge storage period) of high sensitivity image sensor 24 will be described later.

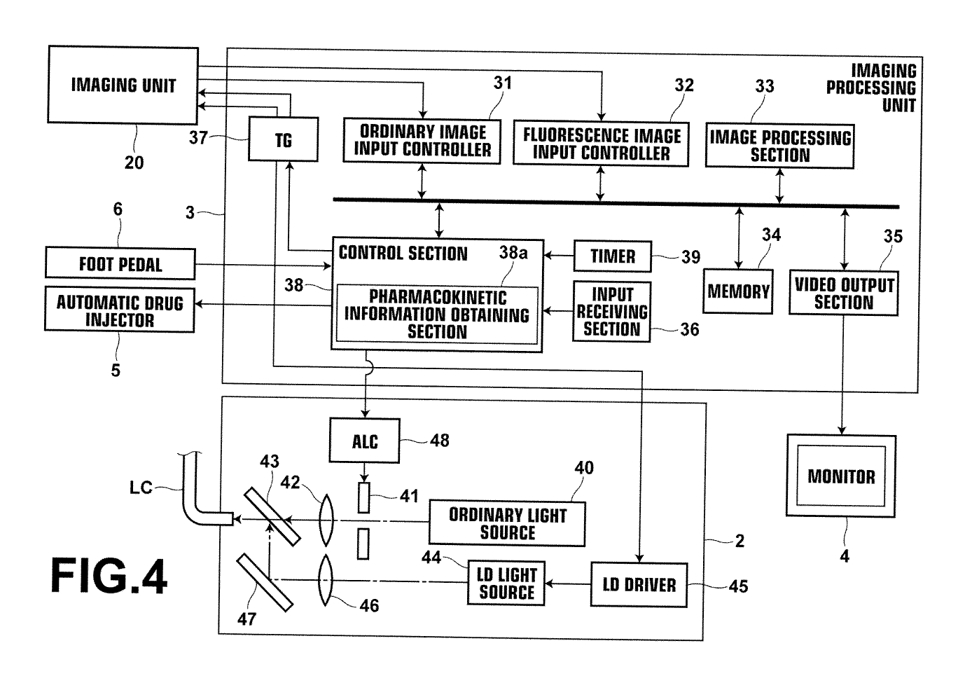

As shown in FIG. 4, image processing unit 3 includes ordinary image input controller 31, fluorescence image input controller 32, image processing section 33, memory 34, video output section 35, input receiving section 36, TG (timing generator) 37, and control section 38.

Ordinary image input controller 31 and fluorescence image input controller 32 are each provided with a line buffer having a predetermined capacity and temporarily storing an ordinary image signal or a fluorescence image signal for each frame outputted from imaging control unit 27 of imaging unit 20. Then, the ordinary image signal stored in ordinary image input controller 31 and the fluorescence image signal stored in fluorescence image input controller 32 are stored in memory 34 via the bus.

Image processing section 33 receives ordinary image signals and fluorescence image signals for one frame read out from memory 34, then performs predetermined processing on these image signals, and outputs the resultant image signals to the bus.

Video output section 35 receives the ordinary image signal and fluorescence image signal outputted from image processing section 33 via the bus, generates a display control signal by performing predetermine processing on the received signals, and outputs the display control signal to monitor 4.

Input receiving section 36 receives input from the operator, such as patient information related to a patient (subject), various types of operation instructions, and control parameters. TG 37 outputs drive pulse signals for driving high sensitivity image sensor 24 and image sensor 26 of imaging unit 20, and LD driver 45 of light source unit 2, to be described later.

Although the main purpose of control section 38 is to control the overall system, the section further includes pharmacokinetic information obtaining section 38a for obtaining pharmacokinetics of fluorescent agent administered to a patient. In the present embodiment, pharmacokinetic information obtaining section 38a obtains patient information inputted through input receiving section 36 as the pharmacokinetic information of fluorescent agent. In the present embodiment, age, weight, gender, observation region, and the like are obtained as the patient information. But the patient information is not limited to those described above, and any information as long as it is related to pharmacokinetics of a fluorescent agent within the body of a patient may be used as the patient information.

As shown in FIG. 4, light source unit 2 includes ordinary light source 40 that emits ordinary light (white light) L1 having a broad wavelength range from about 400 to 700 nm, condenser lens 42 that condenses the ordinary light L1 emitted from ordinary light source 40, and dichroic mirror 43 that transmits the ordinary light L1 condensed by condenser lens 42 and reflects excitation light L2, to be described later, thereby inputting the ordinary light L1 and excitation light L2 to an input end of the optical cable LC. As for ordinary light source 40, for example, a xenon lamp is preferably used. Aperture 41 is provided between ordinary light source 40 and condenser lens 42, and the aperture value thereof is controlled based on a control signal from ALC (automatic light control) 48.

Light source unit 2 further includes LD light source 44 that emits excitation light L2 having a visible to near infrared wavelength in the range from 700 to 800 nm, LD driver 45 that drives LD light source 44, condenser lens 46 that condenses the excitation light L2 emitted from LD light source 44, and mirror 47 that directs the excitation light L2 condensed by condenser lens 46 toward dichroic mirror 43.

Since ICG is used as the fluorochrome, near infrared light of 750 nm to 790 nm is used as the excitation light L2 in the present invention, but the excitation light L2 is not limited to the light in the wavelength range described above, and is determined appropriately according to the type of fluorochrome or the type of living tissue for causing autofluorescence.

LD driver 45 drives LD light source 44 based on control signals outputted from control section 38 and TG 37 of image processing unit 3, in which control section 38 of image processing unit 3 outputs a control signal to TG 37 based on a signal of depression of foot pedal 6 connected to image processing unit 3 and TG 37 outputs a drive pulse signal to LD driver 45 so that excitation light L2 is emitted from LD light source 44 when foot pedal 6 is depressed.

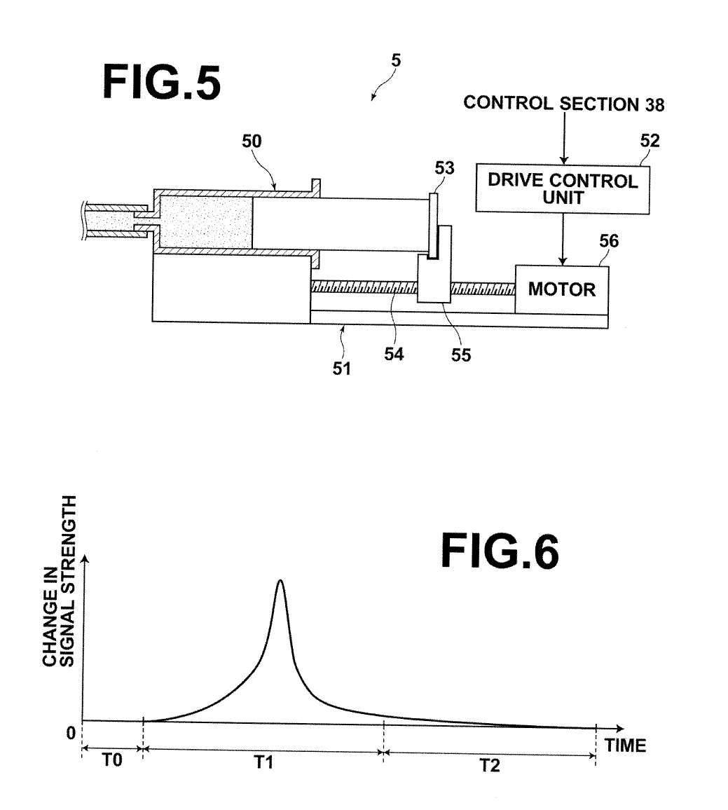

The rigid endoscope system of the present embodiment includes automatic drug injector 5 for automatically injecting a drug into the body of a subject. A schematic configuration of automatic drug injector 5 is shown in FIG. 5. Automatic drug injector 5 includes syringe 50 into which a drug is filled, automatic drug injector body 51 on which syringe 50 is mounted, and drive control unit 52 . Automatic drug injector body 51 includes feed screw 54 for moving plunge 53 of syringe 50, carriage 55, and pulse motor 56. Drive control unit 52 is a unit that outputs a drive pulse signal for driving pulse motor 56 based on a control signal outputted from control section 38 of image processing unit 3.

When syringe 50 filled with a fluorescent agent is mounted on automatic drug injector body 51 and pulse motor 56 is driven by a drive pulse signal from drive control unit 52, carriage 55 is moved by feed screw 54 and plunge 53 is moved, whereby the fluorescent agent is delivered toward the inside of the body of a subject from syringe 50.

Control section 38 of image processing unit 3 outputs a control signal to drive control unit 52 based on a signal of depression of foot pedal 6 connected to image processing unit 3 and drive control unit 52 outputs a drive pulse signal to pulse motor 56 so that the fluorescent agent is delivered toward the inside of the body of a subject when foot pedal 6 is depressed.

Further, image processing unit 3 of rigid endoscope system of the present invention includes timer 39 for measuring an elapsed time from the start of the fluorescent agent administration based on a signal of depression of foot pedal 6 connected to image processing unit 3.

Control section 38 of image processing unit 3 outputs a control signal to imaging unit 20 via TG 37 based on the elapsed time measured by timer 39 to change frame rate of high sensitivity image sensor 24 of imaging unit 20, and outputs a control signal to LD driver 45 of light source unit 2 via TG 37 to control the intensity of excitation light outputted from LD light source 44. The specific control method will be described in detail later.

An operation of the rigid endoscope system of the present embodiment will be described.

First, rigid insertion section 30 with the optical cable LC attached thereto and cable 7 are connected to imaging unit 20 and power is applied to light source unit 2, imaging unit 20, and image processing unit 3 to activate them.

Then, patient information is entered into input receiving section 36 of image processing unit 3 by the operator. The patient information includes age, weight, observation region, and the like of the patient, as described above. The patient information entered into input receiving section 36 is inputted to pharmacokinetic information obtaining section 38a.

Here, the rigid endoscope system of the present embodiment is a system for directing excitation light to fluorescent agent administered to the patient and capturing fluorescence emitted from the fluorescent agent. But, as described above, the concentration of the medicine in the blood changes by one digit or more by the pharmacokinetics of the agent flowing in the bloodstream, causing a problem of insufficient dynamic range of the image sensor for detecting the fluorescence and a signal detected by the image sensor may be saturated. Further, a longer charge storage period of high sensitivity image sensor 24 causes a problem of blurred fluorescence image arising from a pulsation of the observation area.

Consequently, in the rigid endoscope system of the present embodiment, the sensitivity is controlled by controlling the intensity of the excitation light and the charge storage period of high sensitivity image sensor 24 according to the pharmacokinetics of the fluorescent agent described above. More specifically, timing for changing the intensity of the excitation light and the charge storage period of high sensitivity image sensor 24 is obtained by pharmacokinetic information obtaining section 38a based on the entered patient information. Pharmacokinetic information obtaining section 38a includes a lookup table in which three periods, a period T0 from the start of the administration of a fluorescent agent, a period T1 during which the intensity of fluorescence changes largely, and a period T2 during which the intensity of fluorescence is stabilized, are associated with respective patient information. Using the lookup table and entered patient information, pharmacokinetic information obtaining section 38a obtains the periods T0, T1, and T2 described above.

Pharmacokinetic information obtaining section 38a also obtains excitation light intensity control information during the period T1. The excitation light intensity control information may include, for example, excitation light intensity control information predetermined based on the change in the intensity of a fluorescence image signal obtained in the past like that shown in FIG. 6. As shown in FIG. 6, the intensity of the fluorescence image signal gradually increases to a peak and then gradually decreases in the period T1, so that the intensity of the excitation light in the period T1 is reversely controlled in which the intensity is gradually decreased to a bottom and then gradually increased.

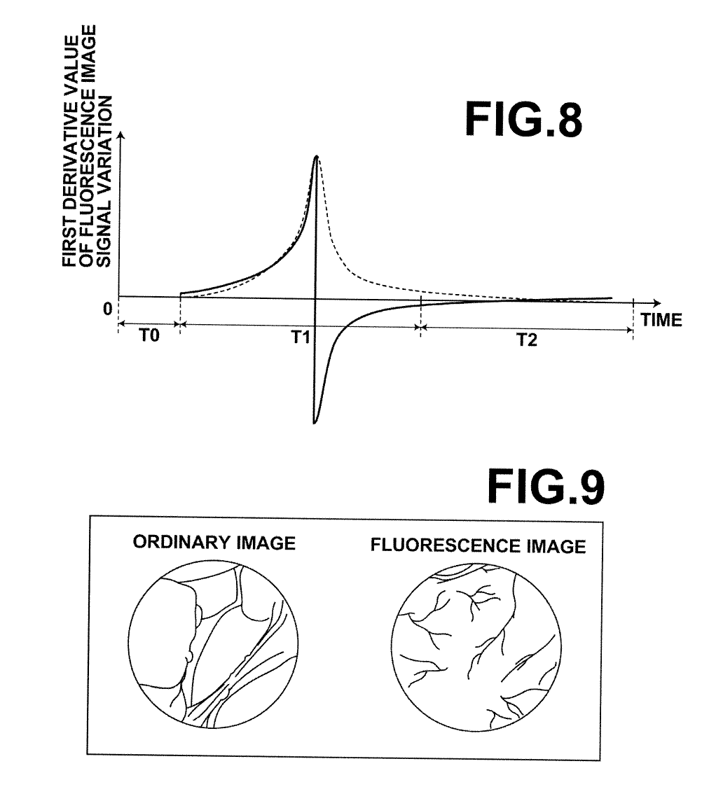

Note that a plurality of types of excitation light intensity control information may be provided in association with patient information and one of them may be selectively set based on the entered patient information. Alternatively, for example, first derivative values of a fluorescence image signal variation (dotted line) obtained in the past shown in FIG. 8 may be obtained, and excitation light intensity control information may be obtained based on the change in the first derivative values (solid line). The method that uses first derivative values described above allows the use of the same excitation light intensity control information for different types of patient information in which fluorescence image signal intensities are different but the way in which the fluorescence image signals change is identical, whereby the number of the types of excitation light intensity control information may be reduced.

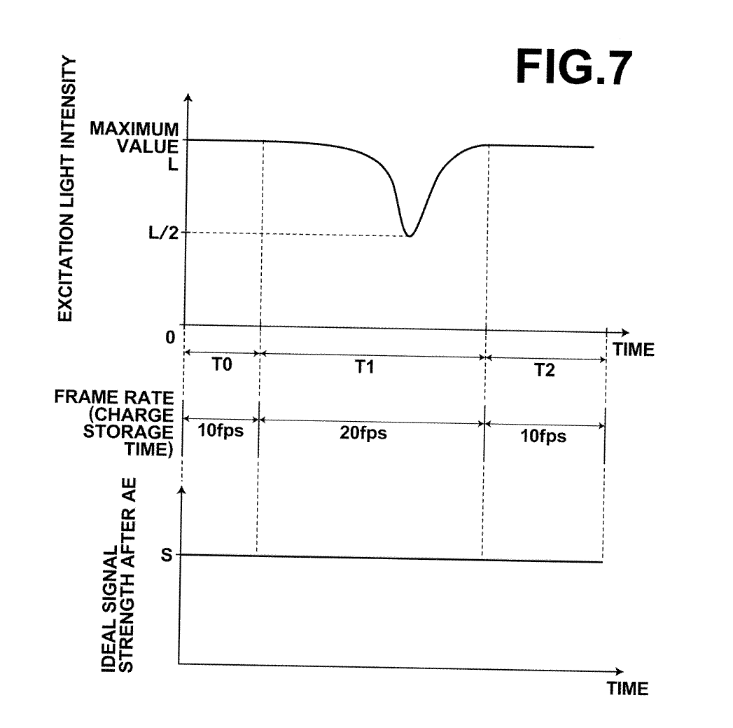

FIG. 7 shows, by way of example, the period TO, period T1, and period T2 obtained in the manner as described above, and a frame rate and excitation light intensity control information in each period. In the present embodiment, high intensity image sensor 24 is controlled at a frame rate of 10 fps in the periods T0 and T2, while in the period Ti it is controlled at 20 fps, as shown in FIG. 7. The excitation light is controlled such that the intensity becomes a maximum vale L in the periods T0 and T2, while in the period T1 it is reduced to L/2 and then increased. The maximum value L of the excitation light is a maximum value within a safety range in which the observation area is not damaged. Ideally, by controlling the frame rate and the excitation light intensity in the manner as described above, the fluorescence image signal outputted from high sensitivity image sensor 24 becomes a constant value, as shown at the bottom of FIG. 7.

The information of period T0, period T1, and period T2, and excitation light intensity control information obtained by pharmacokinetic information obtaining section 38a are set in control section 38 in advance.

Then, rigid insertion section 30 is inserted into a body cavity by the operator and the tip of rigid insertion section 30 is placed adjacent to an observation area.

More specifically, ordinary light L1 emitted from ordinary light source 40 of light source unit 2 is inputted to rigid insertion section 30 through condenser lens 42, dichroic mirror 43, and optical cable LC, and outputted from emission window 30d of rigid insertion section 30, whereby the observation area is irradiated by the light.

An ordinary image based on reflection light reflected from the observation area irradiated with the ordinary light L is captured. More specifically, when ordinary image capturing is performed, an ordinary image L3 based on reflection light reflected from the observation area irradiated with the ordinary light L1 is inputted to insertion member 30b from the tip 30Y thereof, which is guided by the group of objective lenses provided in insertion member 30b and outputted to imaging unit 20.

The ordinary image L3 inputted to imaging unit 20 is reflected by dichroic prism 21 in a right angle direction toward image sensor 26, formed on the imaging surface of image sensor 26 by second image forming system 25. Then, charges are stored in image sensor 26 at a predetermined charge storage period with respect to each frame, and an ordinary image signal according to the stored charges is outputted with respect to each frame.

The ordinary image signals outputted from image sensor 26 are subjected to CDS/AGC (correlated double sampling/automatic gain control) and A/D conversion in imaging control unit 27, and the resultant signals are outputted to image processing unit 3 through cable 7.

The ordinary image signals inputted to image processing unit 3 are subjected to tone correction and sharpness correction in image processing section 33 and stored in memory 34 after being temporarily stored in ordinary image input controller 31. Ordinary image signals read out from memory 34 with respect to each frame are outputted to video output section 35.

Video output section 35 generates a display control signal by performing predetermine processing on the received ordinary image signal, and outputs the display control signal to monitor 4. Then, monitor 4 displays an ordinary image based on the inputted display control signal.

Whereas ordinary image capturing and display are always performed, fluorescence image capturing is started when foot pedal 6 is depressed by the operator.

More specifically, when foot pedal 6 is depressed, a signal of the depression is outputted to control section 38 and a control signal indicating an amount of a predetermined fluorescent agent is outputted from control section 38 to drive control unit 52 of automatic drug injector 5. Then, a drive pulse signal is outputted from drive control unit 52 to pulse motor 56 based on the control signal.

Pulse motor 56 is driven according to the drive pulse signal and plunge 53 is moved, whereby a given amount of the fluorescent agent is delivered toward the inside of the body of the subject.

In the present embodiment, first pass imaging is performed in which a dose of fluorescent agent per administration is reduced to about 1/10 of a normal dose and a process of medicine mass dispersion in blood is imaged. In the first pass imaging, a fluorescence image of a blood vessel before the medicine is dispersed is captured, so that a high signal strength with less background light can be obtained, whereby a high contrast fluorescence image is captured.

In addition to the administration of the fluorescent agent described above, control section 38 starts the operation of the fluorescence image capturing system in response to the depression of foot pedal 6. More specifically, control section 38 outputs a control signal to TG 37 according to the inputted signal of depression of foot pedal 6, and TG 37 outputs drive pulse signals to LD driver 45 of light source unit 2 and high sensitivity image sensor 24 of imaging unit 20 according to the inputted control signal.

Then, according to the inputted drive pulse signal, LD driver 45 drives LD light source 44 to start emission of excitation light L2 and imaging by high sensitivity image sensor 24 of imaging unit 20 is started.

Control section 38 outputs a control signal also to timer 39 to cause timer 39 to start measuring an elapsed time from the start of fluorescent agent administration.

That is, when foot pedal 6 is depressed by the operator, the administration of the fluorescent agent, emission of excitation light L2 onto the observation area, fluorescence image capturing by high sensitivity image sensor 24, and elapsed time measurement by timer 39 are started at substantially the same time.

Then, as shown in FIG. 7, LD light source 44 is controlled such that the intensity of the excitation light L2 becomes the maximum value L and high sensitivity image sensor 24 is controlled to perform fluorescence image capturing at a frame rate of 10 fps from the start of the fluorescent agent administration to the end of the predetermined period T0.

Thereafter, from the end of the period T0 to the end of period T1, LD light source 44 is controlled such that the intensity of the excitation light L2 is gradually decreased from the maximum value L to L/2 and then increased to the maximum value L and high sensitivity image sensor 24 is controlled to perform fluorescence image capturing at a frame rate of 20 fps, i.e., controlled such that the charge storage period with respect to each frame becomes ½ of that of the period T0.

Then, from the end of the period Ti to the end of period T2, LD light source 44 is controlled such that the intensity of the excitation light L2 becomes the maximum value L and high sensitivity image sensor 24 is controlled to perform fluorescence image capturing at a frame rate of 10 fps again, as in the period T0.

Fluorescence image L4 based on fluorescence emitted from the observation area irradiated with the excitation light L2 controlled in the manner as described above is inputted to insertion member 30b from the tip 30Y thereof, which is guided by the group of objective lenses provided in insertion member 30b and outputted to imaging unit 20.

The fluorescence image L4 inputted to imaging unit 20 is formed on the imaging surface of high sensitivity image sensor 24 by first image forming system 23 after passing through dichroic mirror 21 and excitation light cut filter 22. Then, charges are stored in high sensitivity image sensor 24 at a predetermined charge storage period with respect to each frame, and a fluorescence image signal according to the stored charges is outputted with respect to each frame.

The fluorescence image capturing is performed until the time point of the end of the period T2 is measured by timer 39 and control section 38 causes the emission of the excitation light L2 and the imaging by high sensitivity image sensor to be stopped at the time point of the end of the period T2.

Fluorescence image signals outputted from high sensitivity image sensor 24 during a period until the time point of the end of the period T2 are subjected to CDS/AGC (correlated double sampling/automatic gain control) and A/D conversion in imaging control unit 27, and the resultant signals are outputted to image processing unit 3 through cable 7. The fluorescence image signal inputted to image processing unit 3 is subjected to tone correction and sharpness correction in image processing section 33 and stored in memory 34 after being temporarily stored in fluorescence image input controller 32.

Then, fluorescence image signals with respect to each frame are sequentially read out from memory 34 and outputted to video output section 35. Video output section 35 generates a display control signal by performing predetermine processing on the received fluorescence image signal, and outputs the display control signal to monitor 4. Then, monitor 4 displays a fluorescence image based on the inputted display control signal.

Fluorescence images captured in the manner as described above during the time from the start of fluorescent agent administration to the end of the period T2 are displayed on monitor 4 in addition to ordinary images.

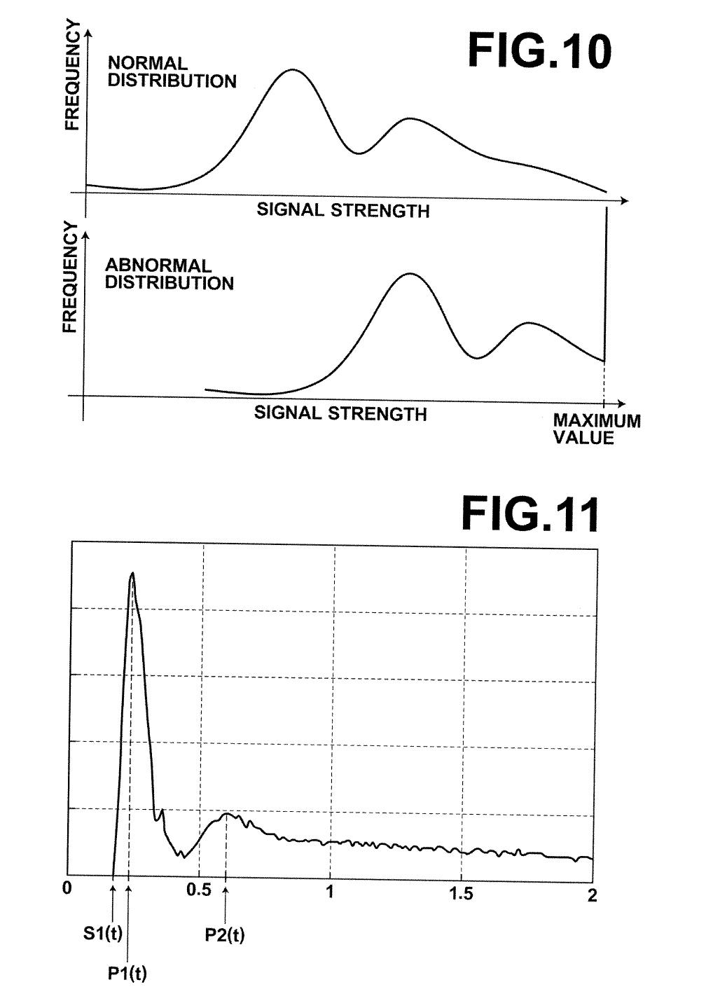

Further, a histogram may be generated using a fluorescence image signal of each pixel of one frame outputted from high sensitivity image sensor 24 and when a frequency of pixels having a maximum fluorescence image signal exceeds a predetermined ratio with respect to total pixels, for example, 30%, the emission of the excitation light may be stopped or blinked. FIG. 10 illustrates example histograms. The upper graph in FIG. 10 shows a histogram obtained when the intensity of the excitation light received by the observation area is normal. On the other hand, the lower graph in FIG. 10 shows a histogram obtained when the intensity of the excitation light received by the observation area is too strong due to, for example, that the tip of rigid insertion section 30 is placed too close to the observation area.

As shown in the lower graph in FIG. 10, when a frequency of pixels having a maximum fluorescence image signal exceeds 30% of total pixels of high sensitivity image sensor 24, the observation area may be prevented from being damaged due to excessive emission of the excitation light by stopping or blinking the excitation light emission.

Further, a fluorescence image signal outputted from high sensitivity image sensor 24 by controlling the charge storage period of high sensitivity image sensor 24 and the intensity of the excitation light in the manner as described above, i.e., the actually obtained fluorescence image signal, and an assumed fluorescence image signal assumed to have been obtained if the intensity of the excitation light and the charge storage period were not controlled. FIG. 11 illustrates, by way of example, an assumed fluorescence image signal.

Then, based on an assumed fluorescence image signal like that shown in FIG. 11, a time S1 (t) when the fluorescence intensity of the observation area is started to change, a time P1 (t) when the fluorescence intensity of the observation area becomes maximum in the first pass, and a time P2(t) when the fluorescence intensity of the observation area becomes maximum in the next pass may be obtained in order to determine whether or not the blood flow is abnormal.

More specifically, for example, the circulating blood volume of an adult male is 75 ml/kg and hence the circulating blood volume for a body weight of 60 kg is calculated as 4500 ml. Meanwhile, the cardiac output of an adult male at rest is 70 ml/s to 80 ml/s, so that the time required for the blood to circulate the entire body once is 54 to 60 seconds. That is, the fluorescent agent administered to the blood passes through the observation area during the time from the start of the administration and 54 to 60 seconds thereafter. Therefore, if the circulation time, P2(t)-P1(t) is shorter than 54 seconds by a predetermined threshold value or longer than 60 seconds by a predetermined threshold value, a determination may be made that the blood flow is abnormal and the determination result may be displayed on monitor 4 in addition to an alert message or the like. It is also possible to make a determination that the blood flow is abnormal if the time required for the fluorescent agent to arrive at the observation area, S1(t), does not fall within a predetermined range.

In the embodiment described above, the ordinary image and fluorescence image are displayed individually, but they may be combined and displayed as a composite image.

More specifically, for example, a blood vessel image signal may be extracted by edge detection or the like based on the fluorescence image signal and the blood vessel image signal may be combined to the ordinary image signal and displayed as a composite image. Alternatively, it is possible that a blood vessel image signal is extracted also from the ordinary image signal, then the blood vessel image signal extracted from the ordinary image signal is subtracted from the blood vessel image signal extracted from the fluorescence image signal, and the subtraction result is combined to the ordinary image signal for display. Generally, the blood vessel image signal in the ordinary image signal represents an image of a blood vessel located in the range from the surface layer of the observation area to a depth of a few tenths of millimeters, while the blood vessel image signal in the fluorescence image signal represents an image of a blood vessel located in the range from the surface layer of the observation area to a depth of several millimeters. Therefore, the blood vessel image signal based on the subtraction result represents an image of a blood vessel located in the depth range from a few tenths of millimeters to several millimeters under fat.

Further, in the embodiment described above, an image sensor in which the charge storage period is changed with the change in the frame rate is used as high sensitivity image sensor 24, but an image sensor in which only the charge storage period is changed without changing the frame rate may be used.

Still further, in the embodiment described above, both the intensity of excitation light and the charge storage period of high sensitivity image sensor 24 are changed, but it is possible that only either one of them is changed.

Further, in the embodiment described above, an arrangement may be adopted in which pharmacokinetic information obtaining section 38a obtains information of a heavily pulsating region of the observation area as the pharmacokinetic information of fluorescent agent and control section 38 fixes the charge storage period of high sensitivity image sensor 24 at a relatively short period when the information of a heavily pulsating region is obtained by pharmacokinetic information obtaining section 38a. More specifically, when lung information is obtained as the region information, for example, the frame rate of high sensitivity image sensor 24 may be fixed at 20 fps in the periods T0 to T2.

Still further, in the embodiment described above, the fluorescence detection sensitivity is controlled by controlling the excitation light intensity and the charge storage period of high sensitivity image sensor 24, but the control method is not limited to this and, for example, a method in which an image sensor having an electron multiplying function is used and the fluorescence detection sensitivity is controlled by controlling the gain of the image sensor based on the pharmacokinetic information of fluorescent agent may be used.

Further, in the embodiment described above, a xenon lamp is used as the ordinary light source, but a high intensity white light source using a GaN semiconductor laser (Trade Name: Micro-White, Nichia Corporation) may be used. The high intensity white light source described above is a light source in which light emitted from a 445 nm semiconductor laser is guided into an optical fiber by an optical lens at one end and white light with a total luminous flux of 50 lm is emitted from the other end of the optical fiber applied with a phosphor material.

Still further, in the embodiment described above, a blood vessel image is extracted, but images representing other tube portions, such as lymphatic vessels, bile ducts, and the like may also be extracted.

Further, in the embodiment described above, the fluorescence image capturing apparatus of the present invention is applied to a rigid endoscope system, but the apparatus of the present invention may also be applied to other endoscope systems having a soft endoscope. Still further, the fluorescence image capturing apparatus of the present invention is not limited to endoscope applications and may be applied to so-called video camera type medical image capturing systems without an insertion section to be inserted into a body cavity.

What is claimed is:

1. A fluorescence image capturing method for capturing a fluorescence image by directing excitation light to an observation area of a subject administered with a fluorescent agent, receiving and photoelectrically converting, by an image sensor, fluorescence emitted from the fluorescent agent of the observation area irradiated with the excitation light, and storing charges by the image sensor for a predetermined charge storage period, the method comprising the steps of:
obtaining pharmacokinetic information of the fluorescent agent; and
controlling the intensity of the excitation light and/or the charge storage period of the image sensor based on the obtained pharmacokinetic information of the fluorescent agent.

2. A fluorescence image capturing apparatus, comprising:
an excitation light emission unit for emitting excitation light which is directed to an observation area of a subject administered with a fluorescent agent;
an imaging unit having an image sensor for receiving and photoelectrically converting fluorescence emitted from the fluorescent agent of the observation area irradiated with the excitation light and capturing a fluorescence image by storing charges for a predetermined charge storage period by the image sensor;
a pharmacokinetic information obtaining unit for obtaining pharmacokinetic information of the fluorescent agent; and
a control unit for controlling the intensity of the excitation light and the charge storage period of the image sensor based on the pharmacokinetic information of the fluorescent agent obtained by the pharmacokinetic information obtaining unit.

3. The apparatus of claim 2, wherein:
the pharmacokinetic information obtaining unit is a unit that obtains information of a heavily pulsating region of the observation area as the pharmacokinetic information of the fluorescent agent; and
the control unit is a unit that fixes the charge storage period of the image sensor at a relatively short period when the information of a heavily pulsating region is obtained by the pharmacokinetic information obtaining unit.

4. A fluorescence image capturing apparatus, comprising:
an excitation light emission unit for emitting excitation light which is directed to an observation area of a subject administered with a fluorescent agent;
an imaging unit having an image sensor for receiving and photoelectrically converting fluorescence emitted from the fluorescent agent of the observation area irradiated with the excitation light and capturing a fluorescence image by storing charges for a predetermined charge storage period by the image sensor;
a pharmacokinetic information obtaining unit for obtaining pharmacokinetic information of the fluorescent agent; and
a control unit for controlling the intensity of the excitation light based on the pharmacokinetic information of the fluorescent agent obtained by the pharmacokinetic information obtaining unit.

5. The apparatus of claim 2, wherein the control unit is a unit that causes the intensity of the excitation light to become relatively small only for a predetermined period based on the pharmacokinetic information of the fluorescent agent.

6. The apparatus of claim 4, wherein the control unit is a unit that causes the intensity of the excitation light to become relatively small only for a predetermined period based on the pharmacokinetic information of the fluorescent agent.

7. The apparatus of claim 5, wherein:
the pharmacokinetic information obtaining unit is a unit that obtains an intensity of the excitation light in the predetermined period based on the pharmacokinetic information of the fluorescent agent; and
the control unit is a unit that controls the intensity of the excitation light based on the intensity of the excitation light in the predetermined period obtained by the pharmacokinetic information obtaining unit.

8. The apparatus of claim 6, wherein:
the pharmacokinetic information obtaining unit is a unit that obtains an intensity of the excitation light in the predetermined period based on the pharmacokinetic information of the fluorescent agent; and
the control unit is a unit that controls the intensity of the excitation light based on the intensity of the excitation light in the predetermined period obtained by the pharmacokinetic information obtaining unit.

9. The apparatus of claim 2, wherein:
the pharmacokinetic information obtaining unit is a unit that obtains timing for changing the intensity of the excitation light based on the pharmacokinetic information of the fluorescent agent; and
the control unit is a unit that controls the intensity of the excitation light based on the timing obtained by the pharmacokinetic information obtaining unit.

10. The apparatus of claim 4, wherein:
the pharmacokinetic information obtaining unit is a unit that obtains timing for changing the intensity of the excitation light based on the pharmacokinetic information of the fluorescent agent; and
the control unit is a unit that controls the intensity of the excitation light based on the timing obtained by the pharmacokinetic information obtaining unit.

11. The apparatus of claim 2, further comprising:
an assumed fluorescence image signal obtaining unit for obtaining an assumed fluorescence image signal assumed to have been outputted from the image sensor if the excitation light intensity control based on the pharmacokinetic information of the fluorescent agent were not performed based on a fluorescence image signal representing a fluorescence image captured by the imaging unit under the excitation light intensity control by the control unit and information of the excitation light intensity control by the control unit; and
an abnormality determination unit for determining an abnormality in the subject based on a change in the assumed fluorescence image signal.

12. The apparatus of claim 4, further comprising:
an assumed fluorescence image signal obtaining unit for obtaining an assumed fluorescence image signal assumed to have been outputted from the image sensor if the excitation light intensity control based on the pharmacokinetic information of the fluorescent agent were not performed based on a fluorescence image signal representing a fluorescence image captured by the imaging unit under the excitation light intensity control by the control unit and information of the excitation light intensity control by the control unit; and
an abnormality determination unit for determining an abnormality in the subject based on a change in the assumed fluorescence image signal.

13. A fluorescence image capturing apparatus, comprising:
an excitation light emission unit for emitting excitation light which is directed to an observation area of a subject administered with a fluorescent agent;
an imaging unit having an image sensor for receiving and photoelectrically converting fluorescence emitted from the fluorescent agent of the observation area irradiated with the excitation light and capturing a fluorescence image by storing charges for a predetermined charge storage period by the image sensor;
a pharmacokinetic information obtaining unit for obtaining pharmacokinetic information of the fluorescent agent; and
a control unit for controlling the charge storage period of the image sensor based on the pharmacokinetic information of the fluorescent agent obtained by the pharmacokinetic information obtaining unit.

14. The apparatus of claim 2, wherein the control unit is a unit that causes the charge storage period to become relatively short only for a predetermined period based on the pharmacokinetic information of the fluorescent agent.

15. The apparatus of claim 13, wherein the control unit is a unit that causes the charge storage period to become relatively short only for a predetermined period based on the pharmacokinetic information of the fluorescent agent.

16. The apparatus of claim 14, wherein:
the pharmacokinetic information obtaining unit is a unit that obtains a charge storage period in the predetermined period based on the pharmacokinetic information of the fluorescent agent; and
the control unit is a unit that controls the charge storage period of the image sensor based on the charge storage period in the predetermined period obtained by the pharmacokinetic information obtaining unit.

17. The apparatus of claim 15, wherein:
the pharmacokinetic information obtaining unit is a unit that obtains a charge storage period in the predetermined period based on the pharmacokinetic information of the fluorescent agent; and
the control unit is a unit that controls the charge storage period of the image sensor based on the charge storage period in the predetermined period obtained by the pharmacokinetic information obtaining unit.

18. The apparatus of claim 2, wherein:
the pharmacokinetic information obtaining unit is a unit that obtains timing for changing the charge storage period based on the pharmacokinetic information of the fluorescent agent; and
the control unit is a unit that controls the charge storage period of the image sensor based on the timing obtained by the pharmacokinetic information obtaining unit.

19. The apparatus of claim 13, wherein:
the pharmacokinetic information obtaining unit is a unit that obtains timing for changing the charge storage period based on the pharmacokinetic information of the fluorescent agent; and
the control unit is a unit that controls the charge storage period of the image sensor based on the timing obtained by the pharmacokinetic information obtaining unit.

20. The apparatus of claim 2, further comprising:
an assumed fluorescence image signal obtaining unit for obtaining an assumed fluorescence image signal assumed to have been outputted from the image sensor if the charge storage period control based on the pharmacokinetic information of the fluorescent agent were not performed based on a fluorescence image signal representing a fluorescence image captured by the imaging unit under the charge storage period control by the control unit and information of the charge storage period control of the image sensor by the control unit; and
an abnormality determination unit for determining an abnormality in the subject based on a change in the assumed fluorescence image signal.

21. The apparatus of claim 13, further comprising:
an assumed fluorescence image signal obtaining unit for obtaining an assumed fluorescence image signal assumed to have been outputted from the image sensor if the charge storage period control based on the pharmacokinetic information of the fluorescent agent were not performed based on a fluorescence image signal representing a fluorescence image captured by the imaging unit under the charge storage period control by the control unit and information of the charge storage period control of the image sensor by the control unit; and
an abnormality determination unit for determining an abnormality in the subject based on a change in the assumed fluorescence image signal.

22. The apparatus of claim 13, further comprising:
an assumed fluorescence image signal obtaining unit for obtaining an assumed fluorescence image signal assumed to have been outputted from the image sensor if the charge storage period control and the excitation light intensity control based on the pharmacokinetic information of the fluorescent agent were not performed based on a fluorescence image signal representing a fluorescence image captured by the imaging unit under the charge storage period control and the excitation light intensity control by the control unit and information of the charge storage period control of the image sensor and the excitation light intensity control by the control unit; and
an abnormality determination unit for determining an abnormality in the subject based on a change in the assumed fluorescence image signal.

23. The apparatus of claim 2, wherein the control unit is a unit that stops or blinks the emission of the excitation light when a predetermined number or more of pixels having a fluorescence image signal greater than or equal to a predetermined threshold value is detected based on a fluorescence image signal of each pixel forming the fluorescence image outputted from the image sensor.

24. The apparatus of claim 2, wherein the pharmacokinetic information of the fluorescent agent is information that includes information of the subject.
